(19) United States
(12) Patent Application Publication
(10) Pub. No.: US 2023/0181646 A1
(43) Pub. Date: Jun. 15, 2023

(54) METHOD OF PRODUCTION OF SPECIALIZED EXOSOMES

(71) Applicants: Raj Jayashankar, San Diego, CA (US); Roger Schechter, San Diego, CA (US)

(72) Inventors: Raj Jayashankar, San Diego, CA (US); Roger Schechter, San Diego, CA (US)

(21) Appl. No.: 17/550,431

(22) Filed: Dec. 14, 2021

(51) Int. Cl. A61K 35/28; A61K 9/00; C12N 5/0775; C12N 5/00; A61P 17/02

(52) U.S. Cl. CPC A61K 35/28; A61K 9/0014; C12N 5/0668; C12N 5/0018; C12N 5/0665; A61P 17/02; C12N 2320/32; C12N 2330/10

(57) ABSTRACT

The present invention provides exosomes that are secreted from mesenchymal stem cells (MSCs) and compositions thereof. The invention also provides techniques to isolate, characterize and optimize manufacturing of such compositions to obtain highly purified and specialized exosomal populations. The compositions comprising populations of such exosomes are used as therapeutic approaches to treat cellular damages and associated conditions, particularly wound healing.

### FIELD OF INVENTION

The disclosure relates to a composition containing exosomes, a method of manufacturing specialized exosomes, and applications thereof.

### BACKGROUND

Stem cells have always been the prime candidates for exosomal production by researchers. Stem cells are easy to expand stably under cell culture conditions and can be differentiated into different lineages by modification of culture conditions. Since other sources of stem cells, e.g., haemopoetic origin, pose a risk of immune reactions and invasivity of cells, the stem cell type selected for most manufacturing methods tends to be mesenchymal stem cells (MSCs). MSCs were isolated initially from bone marrow sources but now are isolated from fetal tissue sources like an umbilical cord, placenta, Chorion, etc.

An issued JP patent 2019535691 assigned to Exo Stem Biotech Limited discloses a method of treating a condition in a patient or a user in need. The method comprises providing an enriched population of placental chorion-derived mesenchymal stem cells, which are therapeutically effective in treating the condition. The RNA under this process is purified using a NanoDrop spectrophotometer.

An issued EP patent 3,277,296 assigned to Stegi-Ra Trust discloses a composition containing extracellular vesicles used for treating celiac disease. The vesicles are derived from chorionic mesenchymal stem cells (Ch-MSCs). The methods broadly cover separation of tissue, Ultracentrifugation, Phase filtration, washing by Phosphate buffered saline (PBS), and incubation with human interferon γ.

Another US patent application, 2021060084, assigned to Mam Holdings Of West Florida, discloses a Pharmaceutical composition of human amniotic fluids that are used for ameliorating autoimmune and inflammatory conditions. The amniotic fluid is decellularized, i.e., cells are removed and other recipients added for use. These compositions also include exosomes derived from amniotic mesenchymal stem cells (MSCs). These exosomes are able to suppress immune pathways associated with autoimmune conditions.

Although the above-mentioned prior art explains the method of producing exosomes of mesenchymal origin using various techniques, it fails to elaborate techniques on producing exosomes that are highly specific in vivo regarding their functioning.

The present invention seeks to provide compositions containing Ch-MSCs that can be applied therapeutically to skin and associated conditions. Along with other epithelial-cell interfaces, the skin is in direct contact with hostile environments outside our body. Therefore immune surveillance in these systems supports a range of passive and active immune defense mechanisms. Unlike the prior art, the Ch-MSCs used herein need to be stable, intact with an excellent safety profile. Physical segregation steps may induce turbulence and disrupt the integrity of Ch-MSCs and/or exosomes isolated.

In addition, there is also a need for methods for producing exosomes that are highly specific in vivo with respect to their functioning. The compositions derived from such population and methods thereof need to be easy to repeat, with consistent end products. This will enable the manufacturing of specialized exosomes on a large scale for therapeutic or other aesthetic uses.

It is apparent now that numerous methods and systems are developed in the prior art that are adequate for different disease and therapeutic applications. However, even though these inventions may be suitable for the specific purposes to which they address, accordingly, they would not be suitable for the purposes of the present invention as heretofore described. Thus, there is a need for high throughput and standardized methods for the production of exosomes that are used for dermal applications.

### SUMMARY

The object of the present invention is to provide a purified exosomes population. The mesenchymal cells MSCs described in the invention express specific sets of a cluster of differentiation (CD) on their surfaces for easy capture and isolation of desired stem cell population to produce exosomes.

The primary objective of the present invention is to provide a method of producing specialized exosomes to treat cells for skin repair. The method includes sampling chorionic tissue by validating through either PCR analysis or serological analysis. Then, collecting cell population from the chorionic tissue, wherein the cell population is of mesenchymal origin.

This is followed by sorting the cell population into a plurality of first cells based on a fluorescence-activated cell sorting (FACS) mechanism. Followed with the plurality of first cells are seeded in a customized cell medium for expansion.

Then the customized cell medium is filtered to isolate one or more exosomes with a size of 0.22 um from the plurality of first cells for validating the one or more exosomes to derive the specialized exosomes by either PCR-based analysis or serological analysis. Then, the specialized exosomes are characterized by in-vitro testing techniques selected from at least one of next-generation sequencing (NGS), Multidimensional Protein Identification Technology (MUDPIT) or Nano-Particle Analysis. Finally, the specialized exosomes are optimized by modifying the customized cell medium and administering the specialized exosomes to a target cell for skin treatment.

The other objective of the present invention is to provide a population of exosomes carrying specific bioactive molecules.

The other objective of the present invention is to provide treatment for cellular damage and regeneration, including wound healing.

The other objective of the present invention is to provide a population of exosomes that targets exclusive target tissue to reduce damage and inflammation.

The other objective of the present invention is to provide MSC derived exosomes as a carrier to transfer/administer bioactive molecules to target cells like an epithelial barrier of the skin to accentuate wound healing.

The other objective of the present invention is to provide exosomes as a safe and effective regenerative therapy for skin/wound healing due to the absence of genetic material in a typical exosomes cargo.

Another objective of the present invention is to provide more stable exosome-based therapy than cell-based therapy. The use of exosomes increases the shelf life of the cargo bioactive molecule, as the exosomes lack DNA and are not living.

Furthermore, it is possible to load higher doses with efficient and targeted action of therapy while using MSC-derived exosomes over traditional cell-based approaches.

Other objectives and aspects of the invention will become apparent from the following detailed description, taken in conjunction with the accompanying drawings, which illustrate, by way of example, the features in accordance with embodiments of the invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

Although the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

### BRIEF DESCRIPTION OF DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 illustrates a complete overview for the manufacturing of the specialized exosomes;

FIG. 2 illustrates the process for the production of specialized exosomes;

FIG. 3 illustrates the process for the optimization of specialized exosomes;

FIG. 4 illustrates the method of production of specialized exosomes to treat cells for skin repair; and

FIG. 5 illustrates the method for producing specialized exosomes for cellular repair and regeneration.

### DETAILED DESCRIPTION OF THE INVENTION

The methods defined in the present disclosure relate to the production of one or more exosomes, particularly exosomes that have the same functional effects on a target cell. The target cells are the cells where functional effects of the specialized exosome cause a therapeutic effect.

In one embodiment of the present invention, the target cells are the cells of the dermis/skin, and the therapeutic effects aid in skin healing and restoration. The methods of the present invention can also be used to produce exosomal populations in the form of a topical composition purely for skin treatments that improves visual characteristics of the skin.

The term “exosome” refers to extracellular vesicles that are membrane-bound and are ultimately pinched off from cells. These vesicles contain cellular secretory products inside them. The vesicles fuse with cell membrane from the inside before releasing into extracellular space-called exosomes. The exosome might interact with distant cells, wherein it brings cellular and behavioral changes in those distant cells by either releasing their content or interacting with cell surface receptors.

The term “stem cell” refers to undifferentiated or partially differentiated cells that do not possess any specialized function in the body. Therefore stem cells have a unique potential to differentiate into various types of cells within the body. Mesenchymal stem cells (MSCs) are cells or used to describe cells that are multipotent in nature. MSCs are able to differentiate into a few cellular types based on in vitro conditions provided during expansion or after implantation in vivo. MSCs are of particular importance in therapeutic applications due to their ability to provide immune-modulatory effects, including inflammation and wound repairs.

The term “chorionic” refers to chorionic villi—any portion or tissue derived thereof. The chorionic tissue referred also includes tissues that are related to other fetal sources, including placenta, amnion, umbilical cord contents. In one embodiment, the tissue source selected for specialized exosome product is chorionic tissue. The MSC derived from chorionic tissue called ch-MSCs show increased proliferation power, decreased recruitment and proliferation of T-cells, and increased ability to induce angiogenesis. These properties are important, especially in the case of using specialized exosomes for cellular repair and regeneration of skin-related conditions. Appropriately developed ECM (extracellular matrix), reorganization of cells involved in clotting/healing, and Blood vessel formation (angiogenesis) is essential to the healthy skin barrier and wound healing after any damaging event (surgery, aging, sun-related damages, etc.).

The term “cluster of differentiation” refers to a cell surface marker that is used as a distinguishing characteristic of cells. There are various types of CDs of which a specific CD is a surface marker that recognizes a specific antigen present on the leucocytes/T-cells surface. The CD antigens are a diverse class of molecules ranging in functions from cell signaling to cell adhesion.

The term “FACS” refers to a type of cell sorting mechanism used during flow cytometry. Fluorescence-activated cell sorting is a technique that uses samples containing a heterogeneous mix of cellular population and segregating and sorting each of the cellular population into different vials for further analysis. The live cells to be separated are flowed as a ‘stream’ through a laser path. Each cellular population containing similar types of cells has different yet unique surface charge characteristics based on their functionality. Therefore, FACS allows the sorting and counting of cells based on both the physical and biological properties of cells. The laser path in this flow cytometry allows cells (as particles) to be separated along different paths due to the surface charge and into different vials. It is also possible to count each particle/cell passing through the laser path for quantitative analysis.

The term “SFM” refers to the medium used for culturing cells, particularly animal cells that do not have any serum component in them. SFM described herein refers to a medium for culturing MSC that does not have contaminants like FBS (Fetal bovine serum), proteins like albumin, and other metabolites. FBS used in media may also contain exosomes that might interfere with the methods for isolating specialized exosomes. Alternatively, FBS might interfere with other downstream steps involved, particularly those of protein-based validation and characterization of specialized exosomes.

The term “DMEM” refers To Dulbecco's Modified Eagle Medium, which is used largely as a basal media to culture animal cells. This forms the basis for the development of the customized medium that contains or omits media components according to specific culture needs. For example, MSC cultures using serum-free DMEM, OptiMEM, or other MEM variants might have supplements to compensate for the lack of nutrients from serum.

The term “PCR” refers to Polymerase Chain reaction. PCR is a method for rapidly amplifying a very minute quantity of nucleic acid sample-DNA. With each cycle of PCR reaction, millions of copies of DNA are generated, which is now easier to detect and analyze further using other techniques like sequencing.

The term “NGS” refers to techniques of next-generation sequencing in which the nucleic acid and/pr protein contents of a cell are used for characterization. In the context of exosomal characterization, NGS techniques can be used to sequence RNA (OR Mrna) contents of an exosome. These sequence data are used for bioinformatics based evaluation of the specialized exosome against RNA or gene sequencing data of known functional effects.

The term “MUDPIT” refers to a chromatography-based proteomic technique. The technique includes the preparation of a peptide mixture from a protein sample and loaded directly onto a triphasic microcapillary column packed with reversed phase, strong cation exchange, and reversed phase HPLC grade materials. The complex peptide mixture is loaded onto the triphasic microcapillary column; further, the column is placed directly in line with a tandem mass spectrometer. The tandem mass spectrometry data generated from a MudPIT run is then searched to determine the protein content of the original sample.

The term “Affinity based purification” refers to isolating cells based on their affinity or biological association with other cells or biomoles. Affinity chromatography is one such technique in which cells are separated based on properties such as magnetic/charge properties, Enzyme substrate interaction, cellular antigen-antibody interaction, Surface receptor-ligand interactions, and so on. These interactions can either be carried on a column or assisted on a Nanoparticle/bead in which the bead or Nanoparticle carries one of the two moieties involved.

The term “cargo” refers to the contents of endovescicles or specialized exosomes used in the present invention. The contents of the specialized endosome include a wide variety of biomolecules that are, in fact, different classes of substances found in cellular functioning. The substances may include any or all of the following-Growth factors, Cell signaling proteins, Cell cycle, and differentiation related substances, metabolic products, Cell adhesion molecules, other cell surface receptors, CDs, surface molecules for compatibility and recognition, and cell surface structures involved in immune-related functioning.

The term “cell adhesion molecule” refers to a class of proteins found on the surface of cells that are involved in the process of cell adhesion either to ECM (Extra cellular matrix) or to a different cell. There are various types of cell adhesion molecules (CAMs) present in cells—Integrins typically bind to the extracellular matrix, while selectins, cadherins, and IgSF are associated with cell-cell adhesion.

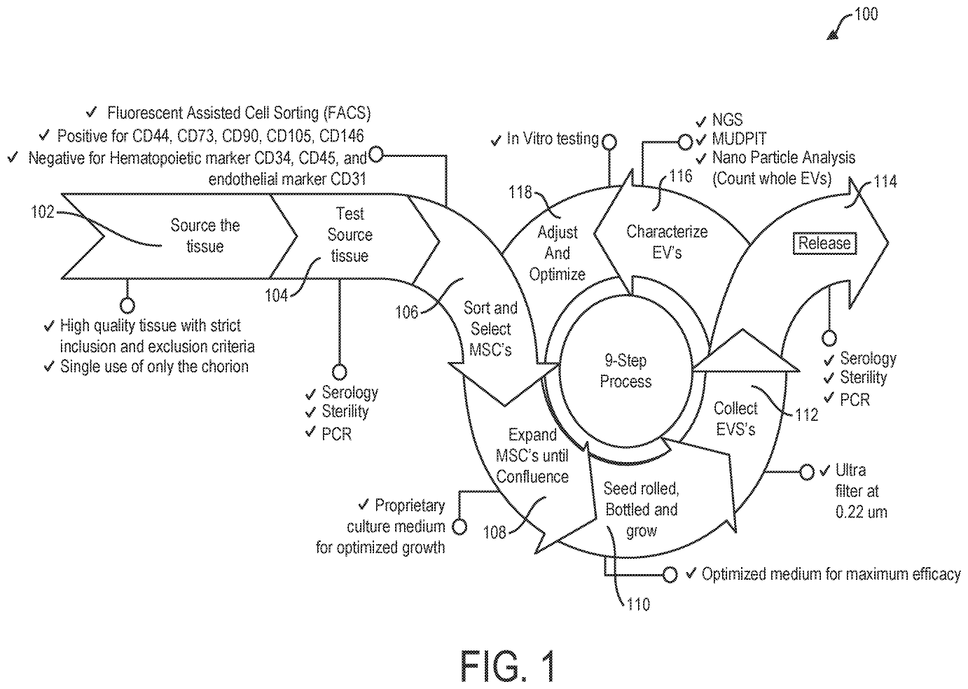

FIG. 1 illustrates a complete overview of the manufacturing of specialized exosomes. The present invention relates to compositions derived from a chorionic source, containing particularly mesenchymal stem cells of adult origin. Such cells are multipotent in nature, i.e., they are able to differentiate into a few cell types found in the body. This flexible differentiation capacity of the MSC is used to generate cell populations of different potential and functioning. Any one of such cell subsets can be used to obtain various exosomal vesicles with corresponding functional aspects.

The invention extends the principle to obtain an exosomal population that is highly consistent in size and functionality. The process of manufacturing such an exosomal population starts with collecting chorionic tissue 102 from a source or biopsy material. There are variations in the quality and composition of tissue collected as sampling. Hence, it is necessary to validate that the cells collected during a tissue biopsy 104 are indeed of mesenchymal origin. Therefore, the source tissue/cells are analyzed using techniques like PCR to verify using sequence similarity or using serological studies.

The cells obtained from chorionic tissue, as a result, might comprise a different type of Mesenchymal stem cells according to cell surface features. In one embodiment, the mesenchymal stem cells are separated according to Cluster of differentiation (CDs) 106 expressed on their cell surface using techniques of cell sorting like Flow cytometry, preferably Fluorescent-activated cell sorting (FACS). A particular mesenchymal stem cell of interest exhibits a pattern of cell surface molecules, in which some CDs are present on the cell surface of these cells. In one embodiment, the mesenchymal stem cell of interest expresses any or all of the following CD44, CD73, CD90, or CD 146. In addition, the mesenchymal stem cells of interest can also be segregated on the basis of exclusion, wherein some CDs are not expressed on their cell surfaces, for example, mesenchymal stem cells devoid of any or all of the following surface markers-hemopoietic marker CD34, CD45 or endothelial marker 31.

The FACS based cell sorting ends in the collection of a first plurality of cells, i.e., mesenchymal stem cells with required expression of CD's as cell surface markers. These cells are now grown and expanded in animal cell cultures in vitro 108. The first plurality of cells, being living cells of animal origin, require stringent nutrient and culture conditions. Of particular importance is the type of culture medium used to propagate the first plurality of cells. The culture medium used can be selected from those known in the art, e.g., DMEM, H.DEMEM, or optiMEM. In one embodiment, it is possible to use a custom-made culture medium according to specific requirements of the first plurality of cells to get efficient cell output. In one of the embodiments, the culture medium used is free of serum, known as serum free medium (SFM). The use of SFM allows disparities to be controlled during the growth of cells, especially wherein the cells are specialized mesenchymal stem cells.

The first plurality of cells is expanded by seeding the cells in a customized cell medium inside a culture bottle 110. The bottles are incubated and rolled according to standard animal culture protocols known in the art. The first plurality of cells can be propagated in either type of cell culture technique—2D cell cultures or 3-D cell cultures.

After the first plurality of cells is successfully established in any animal cell culture setup of choice, the first plurality of cells is provided with such culture conditions or induced exclusively to provide exosomes from the first plurality of cells. All of the exosomes released in the cell culture media are now required to be separated from the mesenchymal stem cells present in the culture chamber. This is achieved by ultra-filtration using a filter (such as a 0.22 um filter) to isolate one or more exosomes of a predefined size to obtain a specific subset of exosomal population from the culture medium 112. These exosomes are collected for further analysis.

The exosomal population collected by filtration is subject to verification by PCR or serological based analysis 114. In one embodiment, at least one of PCR and serological-based techniques validate the identities of both the tissue biopsy/Cell sample (collected at the start of the protocol) and the exosomal population (collected from a seeded and expanded MSC culture).

The PCR based amplification highlights the genetic content of the cells in question. In contrast, Serological tests determine serological markers and their relation to exosomal contents. In some embodiments, an additional quality check of sterility at each of the two steps could also be employed to exclude occurrence and proliferation of contaminants during each of the steps, in which tissue biopsy or cell sample collected at the start of protocol and the exosomal population are ready to be propagated further for expansion and manufacturing.

The method to expand stably and safely those exosomes, which express specific markers on their surfaces in addition to specific contents carried inside the same exosome. These cells are called specialized exosomes, mainly due to highly specific surface characteristics and cargo load. The filtered and verified exosomes obtained at the end might still be inconsistent with reference to their functional effects either in vitro or in vivo.

The method characterizes these exosomal populations to arrive at a consistent batch of specialized exosomes. In one embodiment, the verified exosomes are analyzed by Next-generation sequencing (NGS), proteomics-based chromatography, or affinity-based purification 116. The affinity-based purification can be based on nanobeads, magnetic beads, or cross-linked polymers that associate specifically with cell surface markers of the specialized exosomes. These markers are selected based on the functional effect of the specialized exosome, for example, CD's, Markers with specific immuno-modulatory functions, Cell adhesion molecules that bind to a target cell (like epithelial skin barrier), and so on. In one embodiment, NGS and proteomics based approach called MUDPIT may be used to characterize specialized exosomes. These techniques can be used for qualitative as well as quantitative analysis of contents within an exosome called cargo.

As an added measure and to streamline the overall process of manufacturing the specialized exosomes, in vitro testing of the characterized exosomes can be done according to protocols known in the art. The specialized exosomes can be further studied based on growth characteristics and requirements. The culture medium used for expansion and seeding of MSC (which produced mixed exosomal population), can be fine-tuned to produce batches of an exosomal population that has more numbers of specialized exosomes, making the manufacturing more efficient and time-saving 118. The new version of the customized cell culture medium can be used again for repeated growth and expansion in culture bottles, resulting in a reliable production method for a particular type of exosome having intended in vivo functioning.

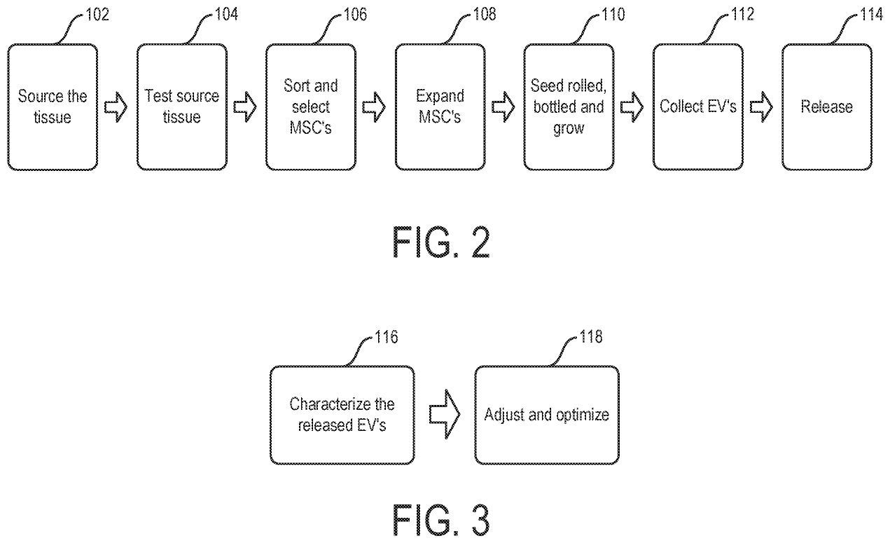

FIG. 2 illustrates the process for the production of specialized exosomes. One skilled in the art will appreciate that placental chorionic cells and the Ch-MSCs derived from them are hypo-immunogenic or non-immunogenic. Furthermore, when exosomes derived out of these Ch-MSCs 102 are used as compositions with conditioned media or ECM are administered, they are also hypo-immunogenic or non-immunogenic. Therefore, manufacturing of such composition can be derived from autologous sources, giving flexibility to the process involved in Tissue/exosome collection.

Fetal Tissues collected from a solid or liquid biopsy are washed with PBS (Phosphate buffered saline). The amnion and chorion are mechanically subdivided into small pieces, and they are subjected to a two-step enzymatic digestion: the cell suspension is then filtered through a filter. The collected cells are analyzed for the presence of Ch-MSCs 104 by subjecting the cell suspension fraction to PCR, wherein the cell constituents are amplified and detected or subjected to serological analysis, and related cell surface moieties are detected. Different Ch-MSCs were also studied to differentiate into cells like keratinocytes, fibroblasts, epithelial cells, or myofibroblasts. These cells are involved in the maintenance and repair of the skin barrier and hence are useful for applications disclosed herein. The purity of all MSC preparations was greater than 95%.

The choice of Ch-MSCs at this step plays an important role in the functional effects of the specialized exosome and applications of compositions that contain these exosomes. A thorough understanding of any one of the functional effects is required beforehand. Any cellular function has a consortium of numerous proteins and the network amongst them to bring about that cellular function (e.g., Wnt/β-catenin signaling involved with skin repair). Other functions (and their protein interaction networks relevant to skin and cell repair) include: the collagen formation pathway, elastin associated microfibrillar proteins, Keratin formation, etc. Therefore, the identification and isolation of biomolecules involved with the expression of such cellular function become important when Specialised exosomes are to be generated for skin treatments.

TABLE 1
summarizes a cellular function involved with skin repair and regeneration
and no. of biomolecules associated with that function.

| | | No. of |
|---|---|---|
| Application | | Biomolecules |
| (Therapeutic/Aesthetic) | Functional/cellular effect | involved |
| Anti-wrinkle/Anti-dryness/ | Collagen formation Pathway | 56 genes |
| Anti-aging/Barrier | Keratin protein | 24 genes + 3 |
| protection | | miRNA |
| | Angiogenesis (Blood vessel | 24 gene + 33 |
| | formation) related protein | miRNA |
| Anti-aging/Barrier | Anti-apoptosis proteins | 3 gene + 1 |
| maintenance | | miRNA |
| Anti-Wrinkle/Barrier | Elastin associated | 33 mRNA/ |
| maintenance/Elasticity | microfibrillar proteins | genes |
| | Fibulin Protein | 5 genes |
| Skin regeneration | Growth factors (multi | 1544 genes |
| | types)—cell proliferation, | |
| | wound healing & tissue | |
| | regeneration | |

The biomolecules mentioned in table 1 can be used as a parameter of choice to isolate Ch-MSCs and/or Specialised exosomes at each step of manufacturing.

These genes or miRNA are involved in the production of cell products that are either enclosed within an exosome and discarded from cytoplasm or Expressed as cell surface receptors, CDs, etc. In any one of the embodiments according to the present invention, the Ch-MSCs are sorted based on CDs expressed on their surfaces. The desired Ch-MSCs are sorted by 106 using a specialized flow cytometry technique called fluorescence-activated cell sorting (FACS) mechanism, wherein the desired MSC expresses cell surface markers—CD44, CD73, CD90, or CD 146. In addition, Ch-MSCs can also be sorted based on the exclusion of certain CDs. For example, Ch-MSCs expressing any or all of the following cell surface markers—hematopoietic marker CD34, CD45, and endothelial marker 31, are removed from the selected batch of Ch-MSCs.

Ch-MSCs selected are seeded and grown in a culture medium till maximum confluence is reached. As a general rule, when a cell line (Ch-MSCs used herein) reaches about 80% confluence, the cells must be subcultured as a different batch into a different culture bottle to ensure proper growth and health of the cells. 80% confluence means when 80% of the surface of a culture vessel is covered with cells.

Ch-MSCs are adult multipotent mammalian cell lines. Such cells must be grown in stringent atmospheric and nutrient conditions, often requiring their own specific growth medium. In one embodiment of the present invention, the Ch-MSC are grown in a customized culture medium that is specific for these cells 108. Its is also possible to grow Ch-MSCs in any commercial or known medium in the art. In either case, the growth conditions and medium are optimized for maximum exosome production 110.

The one or more exosomes generated at the end of the previous step of cell culture are collected based on the size of the exosomes. Exosomes typically consist of two types-one ranging in sizes 30-100 nm and those above 100 nm. Ultra-filtration at 0.22 um or 200 nm is able to separate cell membrane-enclosed nanovesicles and or exosomes from intact Ch-MSCs. The nanovesicles+exosomes collected 112 are separated further to isolate specialized exosomes from the mix. These vesicles end up in the culture medium of the established and seeded Ch-MSC cultures.

The culture medium is tested for sterility and contaminants are removed from the medium. The mix is then validated using PCR protocols and Serology based analysis. This is necessary to check whether the medium collected has specialized exosomal population in it. In one embodiment, the first part of the method results in end products consisting purely or largely specialized exosomes 114.

FIG. 3 illustrates the process for the optimization of specialized exosomes. The two auxiliary steps are followed for the optimization of the conditions for optimum production of the specialized exosomes, to arrive at a therapeutically useful composition.

The end product of the seventh step incorporates mostly-specialized exosomes. These exosomes are distinguished further in a next step to classify the exosomes based on functional effects as described in table 1. The characterization and classification of exosomes 116 into specialized exosomes can be done using highly specific techniques. In one embodiment of the present invention, the techniques used for characterizing exosomes are Next Generation Sequencing (NGS), MUltiDimensional Protein Identification Technology (MUDPIT) or Nano-Particle Analysis.

Nano particle analysis involves purification or isolation of exosomes based on affinity by using capturing beads like nanoparticles, magnetic beads, crosslinked polymer beads, beads coated with antibodies against specific cell surface receptors of exosomes, and so on. NGS involves sequencing data of exosomes and comparing such sequences against known genes/gene products. This validates the relevance of the specialized exosome carrying many important gene products 118.

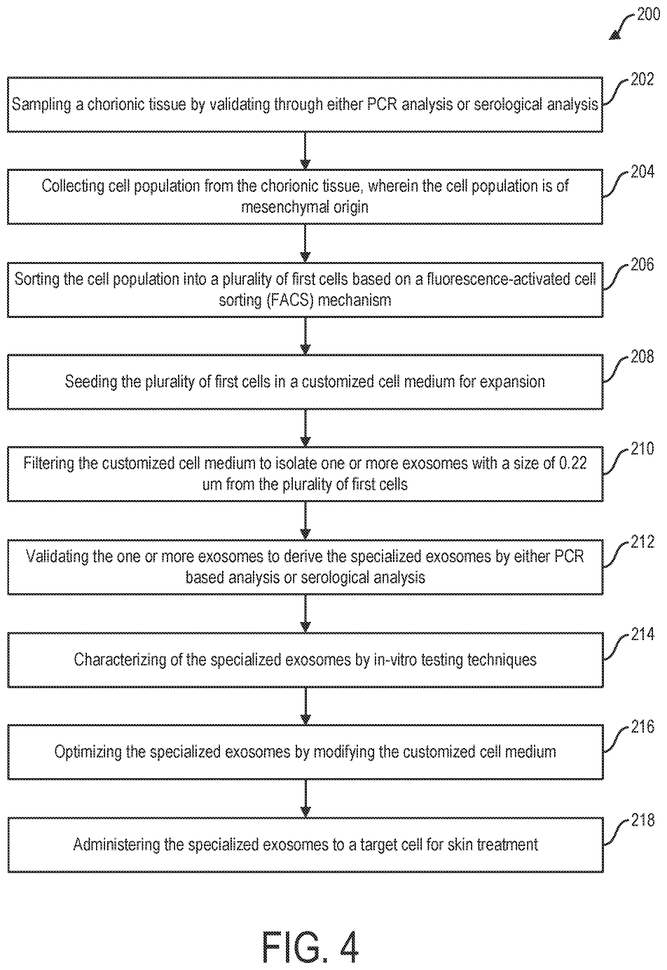

FIG. 4 illustrates the method of production of specialized exosomes to treat cells for skin repair 200. In one embodiment, the method for production of specialized exosomes involves nine technical steps, out of which the last two steps are required for characterization of end products and optimization of the remaining protocol. These steps are needed to fine-tune the method in order to get a stable, consistent, and largely pure population of specialized exosomes, which can be further used for providing compositions for therapeutic/in vivo uses and applications.

According to the current embodiment, the first seven steps involved in the production of specialized exosomes are followed by collecting fetal source tissue sample 204. The cells sampled are exclusively from chorionic tissue 202. This step involves validation of the tissue sample by using various PCR based techniques Or Serological techniques. The purpose of validation is to ascertain that the cells collected are of Ch-MSC type.

The tissue is then disrupted by techniques known in the art to obtain individualized MSCs that can be sorted according to cell surface markers 206 using FACS based flow cytometry. The MSC collected by Flow cytometry are segregated into different vials. The MSC can be grown in the customized culture medium till maximum confluency is achieved.

Further, the MSC sub-type of interest is expanded in cell culture of choice using the customized cell medium adapted according to that subtype of MSC 208. The resultant MSCs release exosomes in the medium which need to be collected for further treatment. This is done using ultra-filtration at 0.22 um diameter size. Finally, the exosomes collected after the filtration step are required to be validated using various PCR based techniques Or Serological techniques 210.

These steps include the first part of the protocol disclosed in the present disclosure involving seven steps, which end with collecting an exosomal population. The population may or may not contain specialized exosomes needed for cellular repair and regeneration. Therefore, the second part of the protocol required following two auxiliary steps.

Firstly, the validated exosomal population is characterized 212 on the basis of functional effects using techniques like NGS, MUDPIT, or Nanoparticle based purification. These techniques collect specialized exosomes from the population of validated exosomes on the basis of surface characteristics or cargo load 214.

Finally, the optimization and modification 216 of the first part of the protocol for high throughput production of specialized exosomes, especially the steps involving customizing the cell medium and size based ultra-filtration. Administering the specialized exosomes to a target cell for skin treatment 218.

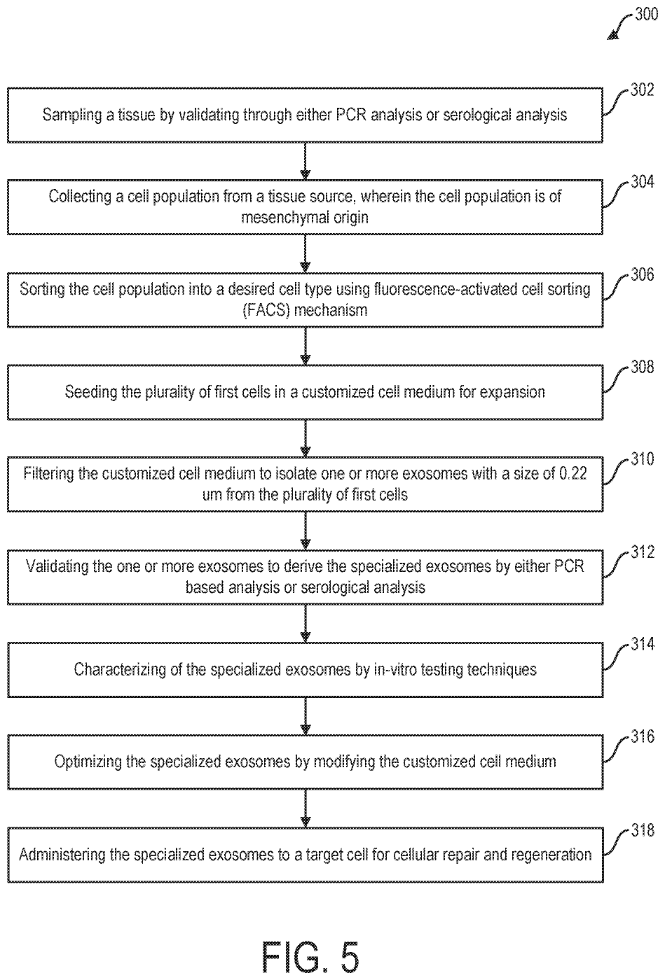

FIG. 5 illustrates the method for producing specialized exosomes for cellular repair and regeneration 300. Firstly, the tissue sampling is done by validating through either PCR analysis or serological analysis 302. Then, collecting a cell population from a tissue source, wherein the cell population is of mesenchymal origin 304.

Sorting the cell population into a desired cell type 306 using fluorescence-activated cell sorting (FACS) mechanism, wherein the desired cell type expresses cell surface markers selected from a group including CD44, CD73, CD90, or CD 146. Then, seeding of the plurality of first cells is performed in a customized cell medium for expansion 308. Filtering of the customized cell medium is followed to isolate one or more exosomes with a size of 0.22 um from the plurality of first cells 310. Validating the one or more exosomes to derive the specialized exosomes by either PCR based analysis or serological analysis 312.

Next, characterizing of the specialized exosomes is performed by in-vitro testing techniques 314. The in-vitro testing techniques are selected from at least one of next-generation sequencing (NGS), proteomics based chromatography, or affinity based purification using a capturing moiety. Optimizing the specialized exosomes by modifying the customized cell medium 316 and lastly, administering the specialized exosomes to a target cell for cellular repair and regeneration 318.

In some embodiments, only segmentation level information is required, and in some embodiment's results of the segmentation module is used as an auxiliary to collect the final results.

From the above, one of ordinary skill in the art will understand sample methods include a method of producing specialized exosomes to treat cells for skin repair. Such method comprises the steps of sampling a chorionic tissue by validating through at least one of PCR analysis or serological analysis; collecting cell population from the chorionic tissue, wherein the cell population is of mesenchymal origin; sorting the cell population into a plurality of first cells based on a fluorescence-activated cell sorting (FACS) mechanism; seeding the plurality of first cells in a customized cell medium for expansion; filtering the customized cell medium to isolate at least one exosome of predefined size from the plurality of first cells; validating the at least one exosome to derive specialized exosomes by at least one of PCR analysis or serological analysis; characterizing the specialized exosomes by in-vitro testing techniques selected from at least one of next-generation sequencing (NGS), Multidimensional Protein Identification Technology (MUDPIT) or Nano-Particle Analysis; optimizing the specialized exosomes by modifying the customized cell medium; and administering the specialized exosomes to a target cell for skin treatment. Such method may characterize the specialized exosomes as being based on at least one functional effect

A similar method provides for producing specialized exosomes for cellular repair and regeneration. The method comprises the step of sampling a tissue by validating through at least one of PCR analysis or serological analysis; collecting a cell population from a tissue source, wherein the cell population is of mesenchymal origin; sorting the cell population into a desired cell type using a fluorescence-activated cell sorting (FACS) mechanism, wherein the desired cell type expresses cell surface marker comprising at least one of CD44, CD73, CD90 or CD 146; seeding the plurality of first cells in a customized cell medium for expansion; filtering the customized cell medium to isolate one or more exosomes of a predefined size from the plurality of first cells; validating the at least one exosomes to derive specialized exosomes using at least one of a PCR analysis or a serological analysis; characterizing of the specialized exosomes by in-vitro testing techniques using at least one of next-generation sequencing (NGS), proteomics based chromatography, or affinity based purification using a capturing moiety; optimizing the specialized exosomes by modifying the customized cell medium; an administering the specialized exosomes to a target cell for cellular repair and regeneration. The functional effects may be either growth and differentiation, immune-modulatory properties, cell signaling, metabolite production or cell surface adhesion. The topical administration is either subcutaneous or trans-dermal and the specialized exosomes are applied to skin for therapeutic or aesthetic purposes. The therapeutical purpose is either repairing damaged skin barrier or restoring functioning of cells, and the aesthetical purpose is either reducing visual appearance of one or more of skin discoloration, edema, scar tissues, redness, inflammation and pre or post surgical complications of dermis.

While the various embodiments of the present invention have been described above, it should be understood that they have been presented by way of example only and not of limitation. Likewise, the figure may depict an example architectural or other configuration for the invention, which is done to aid in understanding the features and functionality that can be included in the invention. The invention is not restricted to the illustrated example architectures or configurations, but the desired features can be implemented using a variety of alternative architecture and configurations. As used in the claims, the definite article “said” identifies required elements that define the scope of embodiments of the claimed invention, whereas the definite article “the” merely identifies environmental elements that provide context for embodiments of the claimed invention that are not intended to be a limitation of any claim.

Although the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

1. A method of producing specialized exosomes to treat cells for skin repair, wherein the method comprising:
sampling a chorionic tissue by validating through at least one of PCR analysis or serological analysis;
collecting cell population from the chorionic tissue, wherein the cell population is of mesenchymal origin;
sorting the cell population into a plurality of first cells based on a fluorescence-activated cell sorting (FACS) mechanism;
seeding the plurality of first cells in a customized cell medium for expansion;
filtering the customized cell medium to isolate at least one exosome of predefined size from the plurality of first cells;
validating the at least one exosome to derive specialized exosomes by at least one of PCR analysis or serological analysis;
characterizing the specialized exosomes by in-vitro testing techniques selected from at least one of next-generation sequencing (NGS), Multidimensional Protein Identification Technology (MUDPIT) or Nano-Particle Analysis;
optimizing the specialized exosomes by modifying the customized cell medium; and
administering the specialized exosomes to a target cell for skin treatment.

2. The method in accordance with claim 1, wherein the characterization of the specialized exosomes is based on at least one functional effect.

3. The method in accordance with claim 2, wherein the at lease one functional effect includes genotypic effect and phenotypic effect.

4. The method in accordance with claim 1, wherein the cell population is of multipotent stem cells.

5. The method in accordance with claim 4, wherein the multipotent stem cells are Mesenchymal stem cells (MSCs).

6. The method in accordance with claim 1, wherein the plurality of first cells does not express a first set of cells surface markers.

7. The method in accordance with claim 1, wherein the first set of cell surface express markers including a hematopoietic marker CD34, a CD45 and endothelial marker 31.

8. The method in accordance with claim 1, wherein the plurality of first cells express a second set of cell surface markers.

9. The method in accordance with claim 8, wherein the second set of surface markers include CD44, CD73, CD90 and CD 146.

10. The method in accordance with claim 1, wherein the plurality of first cells are suspended in the customized cell medium to release the at lease one exosome in extracellular space during expansion.

11. The method in accordance with claim 1, wherein the customized cell medium is either of DMEM, H.DMEM and OptiMeM (Full form).

12. The method in accordance with claim 1, wherein the plurality of first cells grow within the customized cell medium to achieve maximum confluency.

13. The method in accordance with claim 1, wherein the specialized exosomes include at lease one biomolecule to perform specialized functions.

14. The method in accordance with claim 13, wherein the at least one biomolecule include at least one of growth factors or cell signaling proteins.

15. The method in accordance with claim 1, wherein the specialized exosomes are administrated either systemically or topically.

16. A method for producing specialized exosomes for cellular repair and regeneration, the method comprising:
sampling a tissue by validating through at least one of PCR analysis or serological analysis;
collecting a cell population from a tissue source, wherein the cell population is of mesenchymal origin;
sorting the cell population into a desired cell type using a fluorescence-activated cell sorting (FACS) mechanism, wherein the desired cell type expresses cell surface marker comprising at least one of CD44, CD73, CD90 or CD 146;
seeding the plurality of first cells in a customized cell medium for expansion;
filtering the customized cell medium to isolate one or more exosomes of a predefined size from the plurality of first cells;
validating the at least one exosomes to derive specialized exosomes using at least one of a PCR analysis or a serological analysis;
characterizing of the specialized exosomes by in-vitro testing techniques using at least one of next-generation sequencing (NGS), proteomics based chromatography, or affinity based purification using a capturing moiety;
optimizing the specialized exosomes by modifying the customized cell medium; and
administering the specialized exosomes to a target cell for cellular repair and regeneration.

17. The method in accordance to claim 16, wherein the tissue source is either of amnion, chorion, amnion-chorion or umbilical cord.

18. The method in accordance with claim 16, wherein the target cell is one of keratinocytes, fibroblsts, epithelial cells or myofibroblats.

19. The method in accordance with claim 16, wherein one or more biomolecules carried by the specialized exosomes causes functional effects on the target cells.

20. The method in accordance with claim 16, wherein the one or more biomolecules includes at least one of a nucleic acid, a peptide, a protein, a lipid, an antigen, a carbohydrate or a proteoglycan.
